(19) United States
(12) Patent Application Publication
(10) Pub. No.: US 2001/0038836 A1
(43) Pub. Date: Nov. 8, 2001

(54) Application of myeloid-origin cells to the nervous system

(76) Inventors: Matthew During, Philadelphia, PA (US); Paola Leone, Philadelphia, PA (US)

Correspondence Address: CLIFFORD K. WEBER, ESQ, OFFICE OF UNIVERSITY COUNSEL, THOMAS JEFFERSON UNI, 1020 WALNUT STREET, SUITE 620, PHILADELPHIA, PA, 19107, US

(21) Appl. No.: 09/825,713

(22) Filed: Apr. 4, 2001

Related U.S. Application Data
(60) Provisional application No. 60/195,338, filed on Apr. 4, 2000.

(51) Int. Cl. A61K 45/00; C12N 5/08

(52) U.S. Cl. 424/93.7; 435/368

(57) ABSTRACT

The present invention involves the delivery of cells of myeloid origin to a mammalian nervous system and to the use of such cells for treatment of disorders of glial pathology, disorders of neuronal loss or dysfunction, or other disorders, diseases, or trauma involving the nervous system. The invention also includes the delivery of such cells that are transfected with foreign nucleic acid for delivery of potential gene therapy products directly into the CNS.

### CONTINUING APPLICATION DATA

This application claims priority under 35 U.S.C. § 119 based upon U.S. Provisional Application No. 60/195,338 filed Apr. 4, 2000.

### FIELD OF THE INVENTION

The present invention relates to the fields of neurology and cell biology and to a method of delivering mammalian cells of myeloid origin into a mammalian nervous system and, more particularly, to the delivery of hematopoietic stem and progenitor cells into a mammalian nervous system and to the use of such cells for treatment of diseases, disorders and injuries of the nervous system.

### BACKGROUND OF THE INVENTION

Diseases, disorders, and injuries of the nervous system are associated with loss and/or dysfunction of neurons and/or glia. These diseases, disorders, and injuries range from simple monogenetic diseases to complex acquired disorders and trauma. These diseases, disorders, and injuries include, but are not limited to, stroke, Huntington's disease, Alzheimer's disease, Parkinson's disease, amyotrophic lateral sclerosis, brain trauma, spinal cord injury, myelin disorders, immune and autoimmune disorders, metabolic and storage diseases including all of the leukodystrophies and lysosomal storage diseases, and other degenerative, oncological, metabolic, or senescence-related diseases and disorders of the CNS. The neurological damage associated with these conditions is very difficult to treat and/or reverse.

One treatment for neurological damage to the CNS is to replace or restore the function of damaged cells. Neurogenesis in mammals, however, is complete early in the postnatal period. Consequently, the vast majority of cells of the adult mammalian CNS have little or no ability to undergo mitosis and generate new neurons. While a few mammalian species (e.g. rats) exhibit the limited ability to generate new neurons in restricted adult brain regions such as the dentate gyrus and olfactory bulb (Kaplan, J., Comp. Neurol. 195:323, 1981; Bayer, N.Y. Acad. Sci. 457:163, 1985), the generation of new CNS neurons in adult primates does not normally occur (Rakic, Science 227:1054, 1985). This inability to produce new nerve cells in most mammals (and especially primates) may be advantageous for long-term memory retention, however, it is a distinct disadvantage when the need to replace lost neuronal cells arises due to injury or disease.

CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). Degeneration in a brain region known as the basal ganglia can lead to diseases with various cognitive and motor symptoms, depending on the exact location. The basal ganglia consists of many separate regions, including the striatum (which consists of the caudate and putamen), the globus pallidus, the substantia nigra, substantia innominate, ventral pallidum, nucleus basalis of Meynert, ventral tegmental area and the subthalamic nucleus. Many motor deficits are a result of neuronal degeneration in the basal ganglia. Huntington's Chorea is associated with the degeneration of neurons in the striatum, which leads to involuntary jerking movements in the host. Degeneration of a small region called the subthalamic nucleus is associated with violent flinging movements of the extremities in a condition called ballismus, while degeneration in the putamen and globus pallidus is associated with a condition of slow writhing movements or athetosis. Other forms of neurological impairment can occur as a result of neural degeneration, such as cerebral palsy, or as a result of CNS trauma, such as stroke and epilepsy.

In recent years neurodegenerative disease has become an important concern due to the expanding elderly population which is at greatest risk for these disorders. These diseases, which include Alzheimer's Disease and Parkinson's Disease, have been linked to the degeneration of neuronal cells in particular locations of the CNS, leading to the inability of these cells or the brain region they are in to carry out their intended function. In the case of Alzheimer's Disease, there is a profound cellular degeneration of the forebrain and cerebral cortex. In addition, upon closer inspection, a localized degeneration in an area of the basal ganglia, the nucleus basalis of Meynert, appears to be selectively degenerated. This nucleus normally sends cholinergic projections to the cerebral cortex that are thought to participate in cognitive functions, including memory. In the case of Parkinson's Disease, degeneration is seen in another area of the basal ganglia, namely the substantia nigra par compacta. This area normally sends dopaminergic connections to the dorsal striatum which are important in regulating movement. Therapy for Parkinson's Disease has centered upon restoring dopaminergic activity to this circuit through the use of drugs.

In addition to neurodegenerative diseases, acute brain injuries often result in the loss of neurons, the inappropriate functioning of the affected brain region, and subsequent behavior abnormalities.

To date, treatment for CNS disorders has been primarily via the administration of pharmaceutical compounds. Unfortunately, this type of treatment has been fraught with many complications including the limited ability to transport drugs across the blood-brain barrier and the drug-tolerance which is acquired by patients to whom these drugs are administered long-term. For instance, partial restoration of dopaminergic activity in Parkinson's patients has been achieved with levodopa, which is a dopamine precursor able to cross the blood-brain barrier. However, patients become tolerant to the effects of levodopa, and therefore, steadily increasing dosages are needed to maintain its effects. In addition, there are a number of side effects associated with levodopa such as increased and uncontrollable movement.

For a degenerative like Parkinson's Disease, the most comprehensive approach to regain a lost neural function may be to replace the damaged cells with healthy cells. Recently, the concept of neurological tissue grafting has been applied to the treatment of neurological diseases such as Parkinson's Disease. Neural grafts may avert the need not only for constant drug administration, but also for complicated drug delivery systems that arise due to the blood-brain barrier. However, there are limitations to this technique as well. First, cells used for transplantation that carry cell surface molecules of a differentiated cell from another host can induce an immune reaction in the host. In addition, the cells must be at a stage of development where they are able to form normal neural connections with neighboring cells. For these reasons, initial studies on neurotransplantation centered on the use of fetal cells. Several studies have shown improvements in patients with Parkinson's Disease after receiving implants of CNS tissue obtained from 6 to 9 week old human abortuses. Implants of embryonic mesencephalic tissue containing dopamine cells into the caudate and putamen of human patients was shown by Freed, et al. (N. Engl. J. Med. 327:1549-1555 (1992)) to offer long-term clinical benefit to some patients with advanced Parkinson's Disease. Similar success was shown by Spencer, et al. (N. Engl. J. Med. 327:1541-1548, 1992). Widner, et al. (N. Engl. J. Med. 327:1556-1563, 1992) have shown long-term functional improvements in patients with MPTP-induced Parkinsonism that received bilateral implantation of fetal mesencephalic tissue. Perlow, et al. (Science 204:643-647, 1979) describe the transplantation of fetal dopaminergic neurons into adult rats with chemically induced nigrostriatal lesions. These grafts showed good survival, axonal outgrowth and significantly reduced the motor abnormalities in the host animals.

While the studies noted above are encouraging, the use of large quantities of aborted fetal tissue for the treatment of disease raises ethical considerations and political obstacles. There are other considerations as well. Fetal CNS tissue is composed of more than one cell type and, thus, is not a well-defined source of tissue. In addition, there are serious doubts as to whether an adequate and constant supply of fetal tissue would be available for transplantation. For example, in the treatment of MPTP-induced Parkinsonism (Widner, supra) tissue from 6 to 8 fresh fetuses was required for implantation into the brain of a single patient. There is also the added problem of the potential for contamination during fetal tissue preparation. Moreover, the tissue may already be infected with a bacteria or virus, thus requiring expensive diagnostic testing for each fetus used. However, even diagnostic testing might not uncover all infected tissue. For example, the diagnosis of HIV-free tissue is not guaranteed because antibodies to the virus are generally not present until several weeks after infection. Also, only about 5 to 10% of dopaminergic neurons survive, apparently because of adverse immune reaction to the same (Lopez-Lozano, et al., Transp. Proc. 29:977-980, 1997) and because the fetal tissue is primarily dependent on lipid instead of glycolytic metabloism (Rosenstein, Exp. Neurol. 33:106, 1995). For these reasons, attempts have been made to develop alternative cells such as fibroblasts (Kang, et al., J. Neurosci. 13:5203-5211, 1993), fetal astrocytes (Anderson, et al., Int. J. Dev. Neurosci. 11:555-568, 1993), and sertoli cells (Sanberg, et al, Nature Med. 3:1129-1132, 1997) that are suitable for neurotransplantation.

While currently available transplantation approaches represent a significant improvement over other available treatments for neurological disorders, they suffer from significant drawbacks. The inability in the prior art of the transplant to fully integrate into the host tissue, and the lack of availability of neuronal cells in unlimited amounts from a reliable source for grafting are, perhaps, the greatest limitations of neurotransplantation. In order to treat diseases or conditions of the CNS by transplantation, donor cells should be easily available, capable of rapid expansion in culture, immunologically inert, capable of long term survival and integration in the host brain tissue, and amenable to stable transfection and long-term expression of exogenous genes. (Bjorklund, Nature 362:414-415, 1993; Olson, Nature Med. 3:1329-1335, 1997).

The role of stem cells in the adult is to replace cells that are lost by natural cell death, injury or disease. Until recently, the low turnover of cells in the mammalian CNS together with the inability of the adult mammalian CNS to generate new neuronal cells in response to the loss of cells following injury or disease had led to the assumption that the adult mammalian CNS does not contain multipotent neural stem cells. The critical identifying feature of a stem cell is its ability to exhibit self-renewal. The simplest definition of a stem cell would be a cell with the capacity for self-maintenance. A more stringent (but still simplistic) definition of a stem cell is provided by Potten and Loeffler (Development 110:1001, 1990) who have defined stem cells as “undifferentiated cells capable of (a) proliferation, (b) self-maintenance, (c) the production of a large number of differentiated functional progeny, (d) regenerating the target tissue after injury, and (e) a flexibility in the use of these options.”

The ultimate task in replacing CNS cell populations is to select cells capable of differentiating into the post-mitotic and terminally differentiated cells of the nervous system. Several investigators have shown that neural progenitor or stem cells isolated and characterized in vitro may differentiate and result in functional recovery in disease models following in vivo transplantation into the brain, cerebroventricular/cerebrospinal fluid, and/or spinal cord. This work has been reviewed by Snyder, et al. (Adv. Neurol. 72:121-132, 1997) and Fricker, et al. (J. Neurosci. 19:5990-6005, 1999).

Recently, investigators have shown that “brain may turn to blood”, i.e. that neural stem cells delivered into the peripheral blood in vivo differentiate into myeloid cells identical to the lineages obtained with hematopoietic progenitors (Bjornson, C. R., et al., Science 283:534-537, 1999). Similarly, immature hematopoietic cells are able to migrate into brain parenchyma (Ono, K., et al., Biochem. Biophys. Res. Commun. 262:610-614, 1999). When transplanted peripherally (intravenously) into recipient animals, such marrow-derived progenitor cells differentiate into micro- and macro-glia (Eglitis, M. A. and Mezey, E., Proc. Natl. Acad, Sci. USA 94:40805, 1997). Furthermore, a marker of myeloid progenitor cells, CD34, has been shown to be expressed in some cells in the adult brain. (Lin, G., et al., Eur. J. Immunol. 25:1508-1516, 1995). These studies, along with others by Bjornson, et al., suggest that the blood and brain share a common ancestor and that neural stem cells are pluripotent. (Science 283:534-537, 1999).

Given the paucity of successful treatments for diseases, disorders and conditions of the CNS, there remains a need for additional methods of treating patients affected by a disease, disorder, or condition of the CNS. The present invention satisfies this need and overcomes the deficiencies of the prior art treatments by using hematopoietic stem cells derived from bone marrow, from mobilized peripheral blood of humans, from umbilical cord blood, and/or from fetal liver, for delivery in the CNS for the treatment of various diseases.

### SUMMARY OF THE INVENTION

The present invention is a method of targeted delivery of mammalian stem cells of myeloid origin into a nervous system of a mammal, comprising administration of a therapeutically effective amount of said mammalian stem cells of myeloid origin directly into the nervous system of said mammal. The mammalian stem cells of myeloid origin are derived from at least one of the group of bone marrow, mobilized peripheral blood, umbilical cord blood, or fetal liver tissue of a mammal. Administration of these stem cells may occur via any one of a number of methods, including intrathecally, intraventricularly, intracisternally, intraparenchymally into the brain or spinal cord, or systemically. Moreover, administration of said stem cells may occur via a combination of any of these methods.

In one embodiment of the invention the mammalian stem cells of myeloid origin maintain the multipotential capacity to differentiate into neural and glial cells. In another embodiment, the mammalian stem cells of myeloid origin are transiently or stably genetically engineered by at least one viral vector or by non-viral transfection. In still another embodiment, mammalian stem cells of myeloid origin deliver viral vectors, other trransducing agents, or biological pumps of peptides directly into siad nervous system of said mammal. The stem cells delivered via the present invention may be cells expressing CD34 or cells negative for CD34. Other markers may also be used to select appropriate populations of stem cells.

Another aspect of the present invention is a method of treating disorders or diseases of, or trauma to, a nervous system of a mammal, comprising administration of a therapeutically effective amount of mammalian stem cells of myeloid origin into the nervous system of said mammal. The administered mammalian stem cells of myeloid origin migrate from the injection to the site of the damaged nervous system tissue in said mammal, where they are engrafted into the nervous system of said mammal and differentiate into neuronal and glial cells, thereby replacing damaged nervous system tissue of said mammal. The mammalian stem cells of myeloid origin are derived from at least one of the group of bone marrow, mobilized peripheral blood, umbilical cord blood, and/or fetal liver tissue of a mammal. Administration of these stem cells may occur via any one of a number of methods, including intrathecally, intraventricularly, intracisternally, intraparenchymally into the brain or spinal cord, and/or systemically. Moreover, administration of said stem cells may occur via a combination of any of these methods.

In one embodiment of the invention, the mammalian stem cells of myeloid origin are transiently or stably genetically engineered by at least one viral vector or non-viral transfection. In still another embodiment, mammalian stem cells of myeloid origin deliver viral vectors, other trransducing agents, or biological pumps of peptides directly into said nervous system of said mammal. The stem cells delivered via the present invention may be cells expressing CD34 or cells negative for CD34. Other markers may also be used to select appropriate populations of stem cells.

Still another object of the present invention is a method of treating a nervous system disorder, disease, or trauma in a mammal, comprising administration of a therapeutically effective amount of mammalian stem cells of myeloid origin into the nervous system of said mammal, wherein said mammalian stem cells are transiently or stably genetically engineered by at least one viral vector or by non-viral transfection. The administered mammalian stem cells of myeloid origin migrate from the injection to the site of the damaged nervous system tissue in said mammal, where they are engrafted into the nervous system of said mammal and differentiate into neuronal and glial cells, thereby replacing damaged nervous system tissue of said mammal.

Another aspect of the present invention is a method of treating a nervous system disorder, disease, or trauma in a mammal, comprising administration of a therapeutically effective amount of mammalian stem cells of myeloid origin into the nervous system of said mammal, wherein said stem cells of myeloid origin deliver viral vectors, other transducing agents, or biological pumps of peptides directly into said nervous system of said mammal. The administered mammalian stem cells of myeloid origin migrate from the injection to the site of the damaged nervous system tissue in said mammal, where they are engrafted into the nervous system of said mammal and differentiate into neuronal and glial cells, thereby replacing damaged nervous system tissue of said mammal.

### Abbreviations

“CNS” means “central nervous system.”

“KDR” means “kinase tyrosine receptor.”

“FACS” means “fluorescence activated cell sorting.”

“BMMC” means “bone marrow mononuclear cells.”

“GFAP” means “glial fibrillary acidic protein.”

“O-4” means “oligodendrocyte marker O4.”

“FISH” means “Flourescent In Situ Hybridization.”

“MP” means “mix population.”

“LST” means “lysolethicin-hn.”

“NMDA” means “N-methyl-D-aspartate.”

“AAV” means “adenoviral associated vector.”

“GFP” means “green fluorescent protein.”

### Definitions

“genetically-engineered cell” refers to a cell into which a foreign (i.e., non-naturally occurring) nucleic acid, e.g., DNA, has been introduced. The foreign nucleic acid may be introduced by a variety of techniques, including, but not limited to, calcium-phosphate-mediated transfection, DEAE-mediated transfection, microinjection, retroviral transformation, protoplast fusion, and lipofection. The genetically-engineered cell may express the foreign nucleic acid in either a transient or long-term manner. In general, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

### BRIEF DESCRIPTION OF THE DRAWINGS

FIG. 1. A graph of apomorphine-induced rotation over time, showing the effect of stem cell dose response in Parkinsonian rats (normalized to baseline). Rotational behavior is induced in lesioned rats by administration of apomorphine. The rats are tested over a period of at least 6 months prior to injection with stem cells in order to obtain consistent baseline rotational rates.

FIG. 2. A confocal image showing a positive signal for human specific HLA antibody. The image shows an immunohistochemical detection of human-specific HLA expression in the thalamus of a rat two (2) months after the rat was lesioned with NMDA followed by injection with CD34⁺ cells. The image shows the survival of the CD34⁺ cells in the injected area, as well as the migration of the CD34⁺ cells toward the lesioned area.

FIG. 3. Images of immunohistochemical detection showing a positive signal for human-specific mitochondrial antibody (A), nestin antibody (a neuron-specific marker) (B), and human-specific HLA double-labeled with human-specific mitochondrial antibody (C) in the rat brain.

FIG. 4. Images of immunohistochemical detection showing a positive signal for human-specific mitochondrial antibody (A) and nestin antibody (B) in the brain of a rat after the rat was lesioned with LST followed by injection with CD34⁺ cells. The image shows the survival of the CD34⁺ cells in the injected area, as well as the migration of the CD34⁺ cells toward the lesioned area.

FIG. 5. Images of immunohistochemical detection showing a positive signal for human-specific mitochondrial antibody (A) and nestin antibody (B) in the brain of a rat after the rat was lesioned with NMDA followed by injection with CD34⁺ cells. The image shows the survival of the CD34⁺ cells in the injected area, as well as the migration of the CD34⁺ cells toward the lesioned area.

FIG. 6. Images of immunohistochemical detection showing a positive signal for human-specific mitochondrial antibody (A) and nestin antibody (B) in the brain of a rat after the rat was lesioned with NMDA followed by injection with CD34⁺ cells. The image shows the survival of the CD34⁺ cells in the injected area, as well as the migration of the CD34⁺ cells toward the lesioned area.

FIG. 7. Fluorescence In Situ Hybridization (FISH) detection of human stem cells, using probes for the human a-satellite gene, in adult rat brains two (2) months after the rats were injected with CD34⁺ cells.

FIG. 8. Imuunohistochemical detection of GFP expression in the neurons of the striatum of a rat injected with stem cells infected 24 hrs prior to their injection with AAV expressing GFP.

### DETAILED DESCRIPTION OF THE INVENTION

### Methods

Marrow Samples

Hematopoietic stem cells and progenitor cells can be derived from a variety of sources including, but not limited to, bone marrow cells, periperal blood, newborn cord blood, and fetal liver. In a preferred embodiment, human bone marrow is obtained from anonymous human donors by aspiration from the iliac crest and standard bone marrow tap procedures. Bone marrow mononuclear cells (BBMC) (150-200 million cells) are isolated using the method described by Ziegler, et al. (Science 285:1553-1558, 1999)

Isolation of Progenitor and Stem Cells

The stem cell population constitutes only a small percentage of the total number of leukocytes in the bone marrow. At the present time, antigens present on stem cells alone, or that also are present on more differentiated progenitors, have not been fully identified. As in mice, one marker that has been indicated as present on human stem cells, CD34, is also found on a significant number of lineage committed progenitors. Another antigen that provides for some enrichment of progenitor activity are the Class II HLA, particularly a conserved DR epitope recognized by a monoclonal antibody designated J1-43. These markers, however, also are found in numerous lineage committed hematopoietic cells. The Thy-1 molecule is a highly conserved protein present in the brain and in the hematopoietic system of rat, mouse and man. These species differentially express this antigen, the true function of which is unknown. The Thy-1 molecule, however, has been identified on rat and mouse hematopoietic stem cells. This protein is also believed to be present on most human bone marrow cells, but may be absent on stem cells. Another marker that has been indicated as present on human hematopoietic stem cells is kinase tyrosine receptor (KDR). (Ziegler, B. L., et al., Science 285:1553-1558, 1999).

Isolation of populations mammalian bone marrow cell populations which are enriched to a greater or lesser extent with pluripotent stem cells can be achieved through the use of these and other markers. For example, monoclonal antibody My-10, which is found on progenitor cells within the hematopoietic system of non-leukemic individuals, is expressed on a population of progenitor stem cells recognized by My-10 (i.e., express the CD34 antigen) and can be used to isolate stem cells for bone marrow transplantation. See Civin, U.S. Pat. No. 4,714,680. My-10 has been deposited with the American Type Culture Collection (Rockville, Md.) as HB-8483 and is commercially available from Becton Dickinson Immunocytometry Systems (“BDIS”) as anti-HPCA 1. However, since using an anti-CD34 monoclonal antibody alone is not sufficient to distinguish between “stem cells,” and the true pluripotent stem cell (B cells (CD19⁺) and myeloid cells (CD33⁺) make up 80-90% of the CD34⁺ population), a combination of monoclonal antibodies must be used to select human progenitor stem cells.

For example, a combination of anti-CD34 and anti-CD38 monoclonal antibodies can be used to select those human progenitor stem cells that are CD34⁺ and CD38⁻. One method for the preparation of such a population of progenitor stem cells is to stain the cells with immunofluorescently labeled monoclonal antibodies. The cells then may be sorted by conventional flow cytometry with selection for those cells that are CD34⁺ and those cells that are CD38⁻. Upon sorting, a substantially pure population of stem cells results. (Becton Dickinson Company, published European Patent Application No. 455,482).

Additionally, negative selection of differentiated and “dedicated” cells from human bone marrow can be utilized to yield a population of human hematopoietic stem cells with fewer than 5% lineage committed cells. See Tsukamoto et al., U.S. Pat. No. 5,061,620. The stem cells that result are characterized as being CD34⁺, CD3⁻, CD7⁻, CD8⁻, CD10⁻, CD14⁻, CD15⁻, CD19⁻, CD20⁻, CD33⁻, Class II HLA⁺, Thy-1⁺, and KDR⁻.

Furthermore, a two-step purification of low density human bone marrow cells by negative immunomagnetic selection and positive dual-color fluorescence activated cell sorting (FACS) can be used to yield a lin⁻/CD34⁺/HLA-DR⁻ cell fraction that is 420-fold enriched in pluripotent stem cells capable of initiating long-term bone marrow cultures over unmanipulated BMMC obtained after Ficoll-Hypaque separation. (C. Verfaillie et al., J. Exp. Med. 172:509, 1990).

In the present invention, the BMMC are purified using both CD34 and KDR antibodies with either FACS or the AmCell CliniMACS system (Sunnyvale, Calif.) according to the method set forth by Schumm, et al. (J. Hematotherapy 8:209-218, 1999). CD34⁻/KDR⁺ stem cells are isolated after removal of megakaryocytes, which also express KDR. From a single moderate bone marrow tap from a human donor a total of 2,000-10,000 stems cells are isolated using this technique.

Culture of Hematopoietic Cells

The population of stem cells is maintained and expanded using an ex vivo culture system that allows for the clonal population and growth of the stem cells. In this system, the hematopoietic cell population is physically supported by a culture substratum such as a microporous hollow fiber on a microporous membrane that maintains the hematopoietic cells and any associated cells in contact with a liquid culture medium, such as a chemically defined medium suitable for maintenance of stem cells. The pores of the membrane or the hollow fibers can vary in size, so long as they allow culture medium and its components to contact the hematopoietic cells, while providing adequate support for the cells. Preferably, the microporous membrane or the hollow fibers are formed of a synthetic polymer, which can be coated with a cell-adherence promoting peptide, such as mammalian (human) collagen, laminin, fibronectin or the subunits thereof possessing the ability to promote hematopoietic cell attachment. For example, such peptides are disclosed in U.S. Pat. Nos. 5,019,546, and 5,059,425.

The hematopoietic cells may be attached to the interior of a microporous tube or hollow fiber, while the stromal cells are maintained in a fixed relationship from the exterior of the tubing, e.g., on the walls of a chamber containing the growth medium.

During culture, the liquid growth medium may be held as a stationary body that envelops both populations of cells, and is preferably about 25-100% exchanged at fixed intervals, e.g., of 8 hrs-14 days, preferably of about 1-10 days. Alternatively, the culture medium can be continuously circulated through a culture chamber that contains the hematopoietic cells and replaced/replenished at a site remote from the culture chamber. Alternatively, stromal conditioned medium may be used for ex vivo expansion of stem cells and progenitors. As used herein, “stromal conditioned medium” is meant to indicate medium that has been exposed to stromal cells, which cells are removed after said exposure.

Stem Cell Transplantion

6-OHDA Lesion and Apomorphine Test

In order to evaluate the effect of hematopoietic stem cell administration in Parkinsonian rats, male Sprague Dawley rats weighing 280-300 g are anaesthetized with ketamine and xylazine (67 and 6.7 mg-kg⁻¹) and lesioned with 12 μg of the neurotoxin 6-OHDA-HBr (Research Biochemicals, Inc.) injected stereotaxically into the right substantia nigra, at a free base concentration of 4 μg/μL (0.9% saline/0.2% ascorbic acid). Stereotactic coordinates, measured in millimeters from lambda, were +3.5 antero-posterior (AP), +2.1 medio-lateral (ML), and −7.1 dorso-ventral (DV) (Watson and Paxinos, Rat Brain Atlas 1998). A Harvard Apparatus microdialysis pump is used to deliver the 6-OHDA at a rate of (0.5 μL/minute) with a 10 μL Hamilton syringe connected to a 30-guage stainless steel cannula with polyethylene tubing. Three weeks after lesioning, the animals are screened for complete lesions with apomorphine (1 mg/kg in 0.9% saline, 0.2% ascorbic acid) using a hemispheric rotometer. (Hefti, F., et al., Pharma., Biochem. & Beh. 12:185-188, 1980). The number of rotations (contralateral minus ipsilateral) are recorded over a five-minute interval (15-20 minutes) after apomorphine administration. A baseline rotation rate is previously established with three tests conducted at one week intervals. Only animals with a consistent rotation rate >5 rotations/min (with less than ˜25% intra-individual variation between tests) are included in the experimental groups.

Animals selected for placement in the experimental groups are anesthetized with ketamine/xylazine (70 mg & 7 mg per kg respectively). When anesthesia is reached, the animals are placed in a Kopf stereotaxic frame and a medial incision is made in the scalp to expose the cranium. Stereotaxic coordinates are measured from the bregma (bregma and lambda are horizontal) and a drill is used to place a burr hole in the skull above the site of injection. Each animal receives an injection volume of 3 ul at the following coordinates: AP −5.3 mm, ML +2.2 mm(L) and DV −7.6 mm (dura). The injection needle is initially lowered to −8.1 mm and then raised to the final injection coordinate of −7.6 mm. (Watson and Paxinos Rat Brain Atlas).

Table 1 outlines the experimental groupings for stem cell injections. A total of 40 Parkinsonian animals are split into 10 groups.

TABLE 1

| | | | Number of |
|---|---|---|---|
| Group | Cell Type | Number of cells | animals |
| A1 | CD34+/KDR+ | 6000 | 4 |
| A2 | CD34+/KDR+ | 2000 | 5 |
| B1 | CD34+/KDR- | 15000 | 2 |
| B2 | CD34+/KDR- | 6000 | 3 |
| B3 | CD34+/KDR- | 2000 | 3 |
| C1 | CD34+ mixed | 100000 | 3 |
| C2 | CD34+ mixed | 6000 | 4 |
| D | CD34− | 6000 | 6 |
| E | CD34−/GPA+ | 6000 | 6 |
| F | Media | equal volume 3 ul | 3 |

Following cell transplantation, the animals are tested for apomorphine induced rotational behavior at 2, 4, 6, 8, 10, 12 and 20-week intervals post-surgery. The number of rotations (contralateral minus ipsilateral) are recorded over a five-minute interval (15-20 minutes) after apomorphine administration (Hefti, F., et al., Pharma., Biochem. & Beh. 12:185-188, 1980).

KDR+ Stem Cells Transplantation into Neonatal and Adult Mice

In one embodiment of the present invention, the KDR⁺ cells are labeled with an inert fluorescent marker, Cell Tracer Vybrant (Molecular Probes, Inc.). The KDR⁺ cells are directly infused into the lateral ventricle and hippocampus of neonatal mice using stereotactic surgery. A total of 1000 cells are administered per mouse.

Four weeks after cell transplantation, the mice are euthanized and the tissue processed using human mitochondria antibody (Chemicon) as described by Gutekunst et al (J. Neuroscience 18:7674-7686, 1998), and Fluorescent In Situ Hybridization (FISH) (infra) as described by Chen et al. (Cancer Genet. Cytogene 63(1):62-69, 1992). Double labeling is used with antibodies to GFAP (an astrocytic marker), O-2 (an oligodendrocytic marker), and NeuN (a neuron-specific marker).

KDR+ Stem Cells Transplantation into Neonatal and Adult Mice Following a Neuronal Injury that Models Human Stroke

In another embodiment, adult mice undergo a local ischemic lesion, modeling a human stroke, by intraparenchymal (hippocampus and striatum) injection of the vasoactive peptide, endothelin-1 (50 pm/μl), which causes an ischemic tissue injury. The KDR⁺ cells are directly infused into the lateral ventricle of untreated neonatal mice and the hippocampus (2 mm from the endothelin-1 injection) of the treated adult mice using stereotactic surgery. A total of 1000 cells are administered per mouse.

Three to eight weeks after cell transplantation, the mice are euthanized and the tissue processed using human mitochondria antibody (Chemicon) and FISH (infra). Double labeling is used with antibodies to GFAP, O-2, and NeuN (supra).

CD34⁺ and/or CD34⁻ and/or in Combination with KDR⁺ and/or KDR⁻ Stem Cells Transplantation into Adult Rats with Demyelinating Injuries and Neurotoxic Injuries

In yet another embodiement, cells in the following groups are selected from the BMMC as described above: CD34⁺/KDR⁺, CD34⁺/mix population (MP), and CD34⁻/KDR⁺. The selected stem cells are infected with adeno-associated viral vector (AAV) expressing green fluorescent protein (GFP) 24 hrs prior to their injection into the subject animal. Alternatively, the selected cells are relabeled with Cell Tracer Vybrant (Molecular Probes, Inc.) 1-4 hrs prior to their being injected.

Administration: Neonates

Neonatal rats and mice pups aged 24-36 hrs are injected intracerebroventricularly with 2 to 4 μl/2,000 to 8,000 stem cells, selective or mixed.

Administration: Adults

In one group of adult rats, a lysolethin lesion is induced following stem cell transplantation. The rats in this group, which weigh between 320 g and 400 g, are injected intracerebroventricularly with stem cells 2 μl in volume with a flow rate of 0.5 μl/min/2,000 cells in the subcortical white matter at coordinates: A-P=−1.3; M-L=1.0; D-V=2.8 from the dura. Following stem cell injection, these same rats are injected with lysolethicin-hn (LST) at 1 μl volume with a flow rate of 0.5 μl/min at coordinates: A-P=−1.3; M-L=3.2; and D-V=3.0. A 2% solution in Hanks' Balanced Salt Solution is used. The LST is in 50 mg/ml with chloroform/methanol at 1:1 at −20° C.).

In another group of adult rats, a N-methyl-D-aspartate (NMDA) lesion is induced following stem cell injection. The rats in this group, which weigh between 320 and 400 g, are injected intracerebroventricularly with stem cells 2 μl in volume at a flow rate of 0.5 μl/min/2,000 cells in the lateral hypothalamus (LH) at coordinates: A-P=1.8; M-L 1.8; D-V=6.5 (from skull). Following stem cell injection, these same rats are injected with NMDA as follows: 30 nanomolar NMDA in 1 μl volume at a flow rate of 0.5 μl/min. Coordinates for the NMDA-induced lesion in the LH are as follows: A-P=−1.8; M-L 1.8; and D-V=8.5 (from skull).

FISH

As noted above, brain tissue from the subject animals is processed using FISH in order to detect gene expression. Coronal brain tissue throughout the injection site sections having a thickness of 5-7 μm are collected on positively charged glass slides and post-fixed in Carnoy's fixative (3:1 Methanol:Acetic acid) for 10 minutes at room temperature. This is followed by pretreatment procedure with protease and pepsin mixture for 15 minutes in a 37° C. water bath and serial dehydration in ascending grades of ethanol from 70% to 100% for three minutes in each. Tissue sections then are washed in 2×SSC in a 73° C. water bath for 5-10 minutes, followed by dehydration in alcohol and a complete air drying. The air-dried samples are placed in denaturation solution (Formamide, 2×SSC) in a 73° C. water bath for 5-10 minutes. Serial dehydration in ascending grades of ethanol from 70% to 100% for three minutes in each is followed by application of the probe mixture-CEP 7 SO (α-satellite) (Vysis, Part #32-130007) or CEP 18 SG (α-satellite) (Vysis, Part #32-132018). Prior to its application, the probe is mixed and placed in a 73° C. water bath for 5 minutes.

After 16 hours of incubation time in a humidifier and incubator at a temperature of 42° C., the tissue sections are rapidly washed in serial washing solutions (2×SSC and 0.1% NP-40; 0.4×SSC and 0.3% NP-40) for 5-10 minutes in the 73° C. water bath, airdried, and coverslipped with DAPI II counterstain.

Immunohistochemistry

Euthanized subject animals are perfused with 1× PBS followed by 4% paraformaldehyde in 1× PBS, pH 7.4. After being immersed in fixative overnight, brains are cryoprotected in ascending concentration of sucrose from 10% to 30% in PBS. Sections of 15-20 μm are cut on cryostat in the coronal plane and collected on positive charged glass slides, three sections per slide, and air dried for at least one hour. Sections of representative brain region/levels are selected and rinsed in 1× PBS. The rinsed sections are washed twice in 1% of Triton in PBS for 10 minutes each washing and then incubated in 1% H₂O₂ in 50% methanol for 15 minutes. After being rinsed in PBS-Triton brain tissue sections are incubated in 5% normal goat serum 2 times for 10 minutes and then incubated overnight at room temperature in primary monoclonal antibody against human mitochondria (1:20, Chemicon). Antibody is diluted in 0.5% normal goat serum in PBS.

Following incubation, the sections are washed with PBS-Triton. After washing, the sections are treated with biotinylated secondary goat anti-mouse antibody (1:250 dilution; Vector) for 3 hours at room temperature, followed by PBS rinses and incubation with avidin-biotin complex (ABC; 1:250 dilution; Vector) for 2 hours.

Immunohistochemical staining is visualized with 0.5 mg/ml 3′3-diaminobenzidine (DAB) in 1× PBS and 0.1% H₂O₂. For control staining, sections are incubated without primary antibody (immunobuffer only), followed by incubation with secondary antibody. Immunostained tissue sections are air dried, dehydrated in ascending concentrations of ethanol, cleared in xyline, and coversliped with non-aqueous mounting medium.

Genetically Engineered Stem Cell Transplantation

The present invention also includes the delivery of stem cells that are transfected with foreign (i.e., heterologous) nucleic acid, e.g., DNA, is introduced into the stem and progenitor cells prior to their delivery into the nervous system. Foreign nucleic acid may be introduced into hematopoietic stem and progenitor cells or their progeny. A hematopoietic stem or progenitor cell or its progeny that harbors foreign DNA is said to be a genetically-engineered cell. The foreign DNA may be introduced using a variety of techniques. In a preferred embodiment, foreign DNA is introduced into the stem or progenitor cells using the technique of retroviral transfection. Recombinant retroviruses harboring the gene(s) of interest are used to introduce marker genes, such as the E. coli β-galactosidase (lacZ) gene, or oncogenes. The recombinant retroviruses are produced in packaging cell lines to produce culture supernatants having a high titer of virus particles (generally 10⁵ to 10⁶ pfu/ml). The recombinant viral particles are used to infect cultures of the stem or progenitor cells or their progeny by incubating the cell cultures with medium containing the viral particles and 8 μg/ml polybrene for three hours. Following retroviral infection, the cells are rinsed and cultured in standard medium (supra). The infected cells are then analyzed for the uptake and expression of the foreign DNA. The cells may be subjected to selective conditions that select for cells that have taken up and expressed a selectable marker gene.

In another preferred embodiment, the foreign DNA is introduced using the technique of calcium-phosphate-mediated transfection. A calcium-phosphate precipitate containing DNA encoding the gene(s) of interest is prepared using the technique of Wigler et al. (Proc. Natl. Acad. Sci. USA 76:1373-1376, 1979). Cultures of the hematopoietic stem or progenitor cells or their progeny are established in tissue culture dishes. Twenty four hours after plating the cells, the calcium phosphate precipitate containing approximately 20 μg/ml of the foreign DNA is added. The cells are incubated at room temperature for 20 minutes. Tissue culture medium containing 30 μM chloroquine is added and the cells are incubated overnight at 37° C. Following transfection, the cells are analyzed for the uptake and expression of the foreign DNA using techniques that are known in the art. The cells may be subjected to selection conditions such as antibiotic reactions that select for cells that have taken up and expressed a selectable marker gene.

Therapeutic Administration of Progenitor/Stem Cells

According to the present invention, the stem cells are suspended in a sterile pharmaceutically acceptable carrier and administered into the CNS of a mammal, including, but not limited to, a pig, cow, dog, but preferably a human subject, at or near a site of injury or disease. Optionally, treatment with stem cells may be combined with local or systemic anti-inflammatory therapy, for instance administration of a steroid such dexamethosone or methylprednisolone, or administration of a non-steroidal anti-inflammatory agent. The present invention contemplates the optional use of a steroid or non-steroidal anti-inflammatory agent at any dose that is effective in the subject to be treated. Such effective doses are well known to those skilled in the art and include, for example, standard-dose therapy, such as systemic methylprednisolone 100 mg daily for a human adult, and high-dose therapy, such as systemic methylprednisolone 1000 mg daily for a human adult.

In a preferred embodiment, the pharmaceutically acceptable carrier is PBS or a culture medium. However, alternative pharmaceutically acceptable carriers will readily be apparent to those skilled in the art, including but not limited to, aqueous solutions, preferably in physiologically compatible buffers such as Hank's solution, Ringer's solution, or physiological saline buffer.

In a preferred embodiment, the stem cells are administered immediately following CNS injury and are introduced at the site of CNS injury. However, the present invention encompasses administration of stem cells at any time following CNS injury or disease and encompasses introduction of the stem cells at or near a site of CNS injury or disease by any neurosurgically suitable technique. The present invention contemplates a variety of techniques for administration of the therapeutic compositions. Suitable routes include, but are not limited to, systemic delivery, including intramuscular, subcutaneous, intramedullary injections, as well as intrathecal, direct intraventricular, intravenous, intraperitoneal, intranasal, intraparenchymal, or intraocular injections, among others, or a combination thereof.

The compositions and methods of the present invention are useful for treating any injury or disease of the CNS that results in or is accompanied by loss and/or dysfunction of neurons and/or glia. The injury or disease may be situated in any portion of the CNS, including the brain, spinal cord, or optic nerve. One example of such injury or disease is trauma, including coup or countercoup injury, penetrating trauma, and trauma sustained during a neurosurgical operation or other procedure. Another example of such injury or disease is stroke, including hemorrhagic stroke and ischemic stroke. Yet another example of such injury or disease are Alzheimer's disease, Parkinson's disease, amyotrophic lateral sclerosis. Still further examples of CNS injury or disease will be evident to those skilled in the art from this description and are encompassed by the present invention. The compositions and methods of the present invention are useful for treating CNS injury or disease that results in loss and/or dysfunction of neurons and/or glia whether or not the subject also suffers from other disease of the central or peripheral nervous system, such as neurological disease of genetic, metabolic, toxic, nutritional, infective or autoimmune origin.

The assessment of the clinical features and the design of an appropriate therapeutic regimen for the individual patient is ultimately the responsibility of the prescribing physician. It is contemplated that, as part of their patient evaluations, the attending physicians know how to and when to terminate, interrupt, or adjust administration due to toxicity, or to organ dysfunctions. Conversely, the attending physicians also know to adjust treatment to higher levels, in circumstances where the clinical response is inadequate, while precluding toxicity. The magnitude of an administrated dose in the management of the disorder of interest will vary with the severity of the condition to be treated, the patient's individual physiology, biochemisty, etc., and to the route of administration. The severity of the condition, may, for example, be evaluated, in part, by standard prognostic evaluation methods. Further, the dose and dose frequency will also vary according to the age, body weight, sex and response of the individual patient.

Results

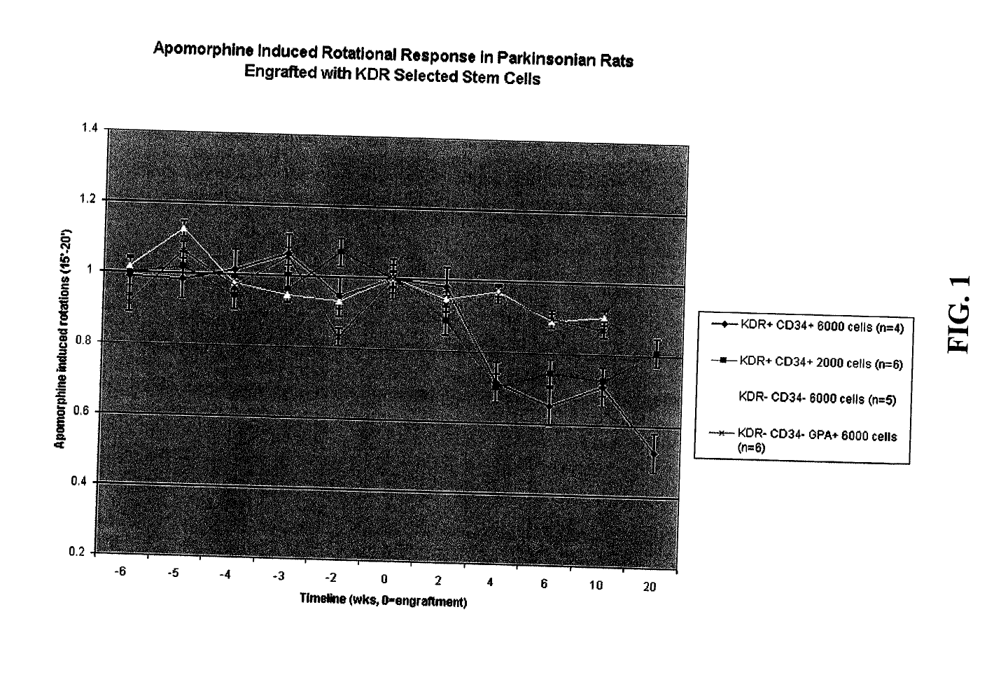

The present invention provides evidence demonstrating that hematopoietic stem and progenitor cells have therapeutic potential when directly administered into the CNS of mammals exhibiting neuronal and/or glial cell loss and/or dysfunction. Parkinsonian rats, i.e., rats lesioned rats by administration of apomorphine, show significant change in rotational rates compared with baseline following treatment with CD34⁺ cells at various concentrations. (FIG. 1) In contrast, Parkinsonian rats injected with CD34⁻ cells, erythroblasts, or the PBS control exhibit no significant change in rotational rate throughout the test period. (FIG. 1)

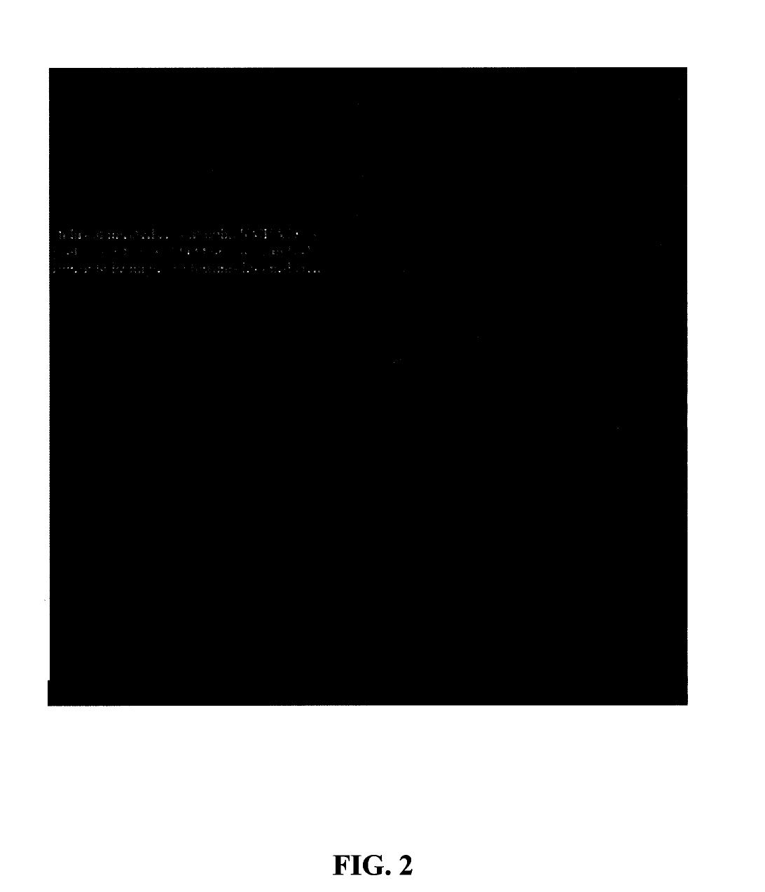

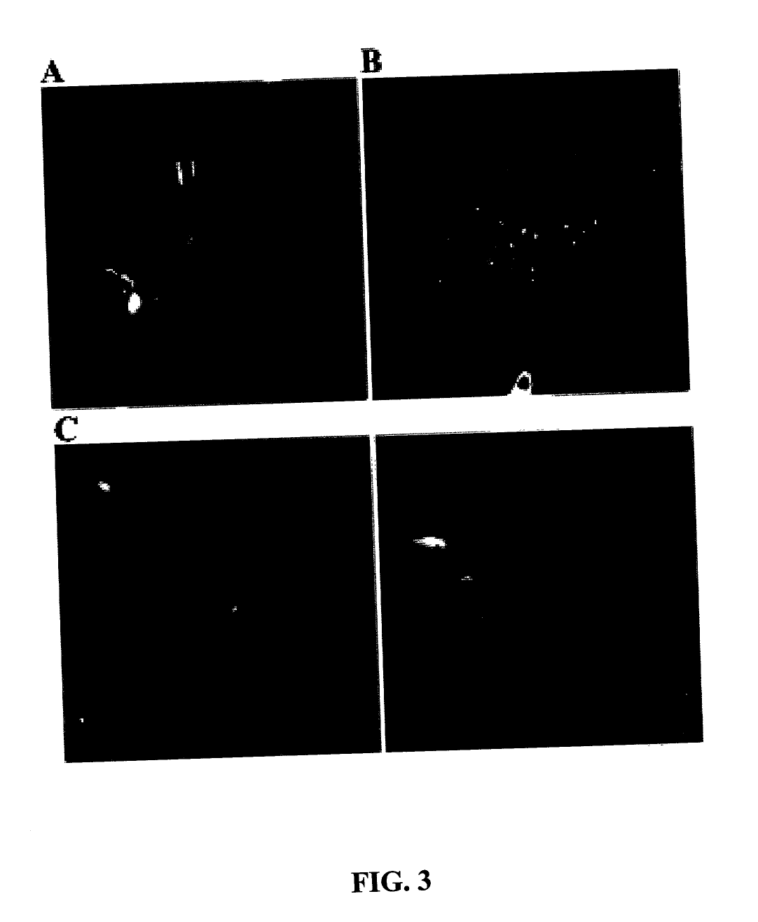

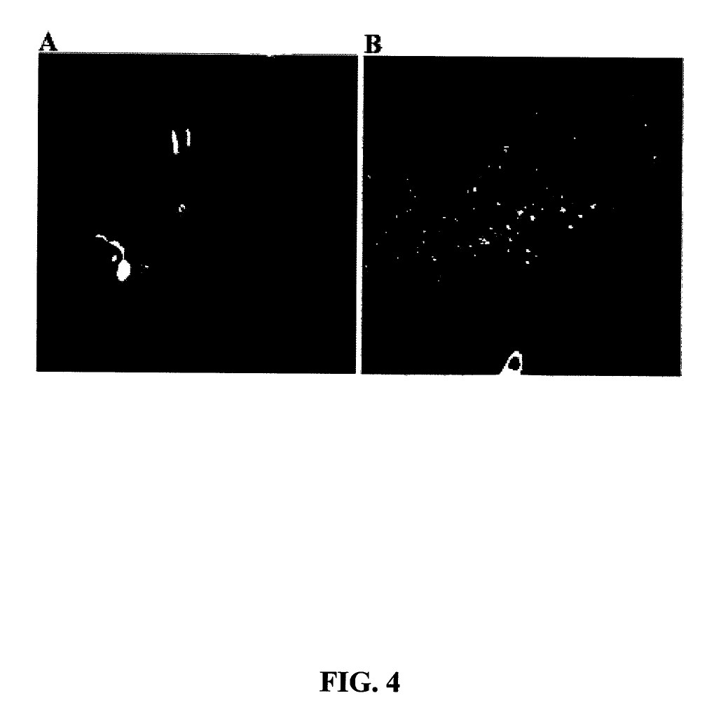

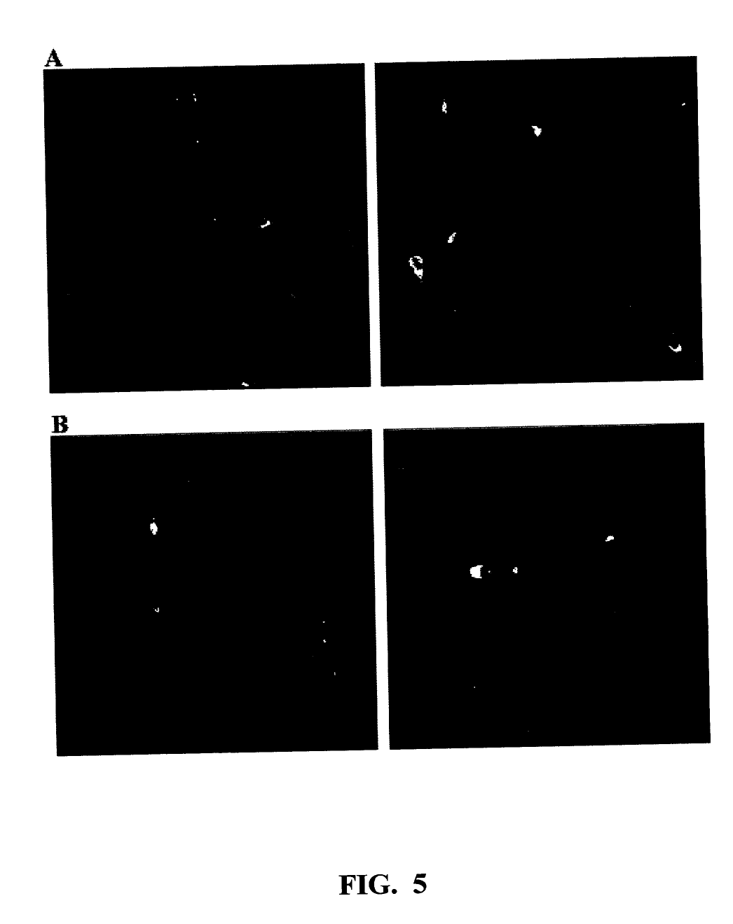

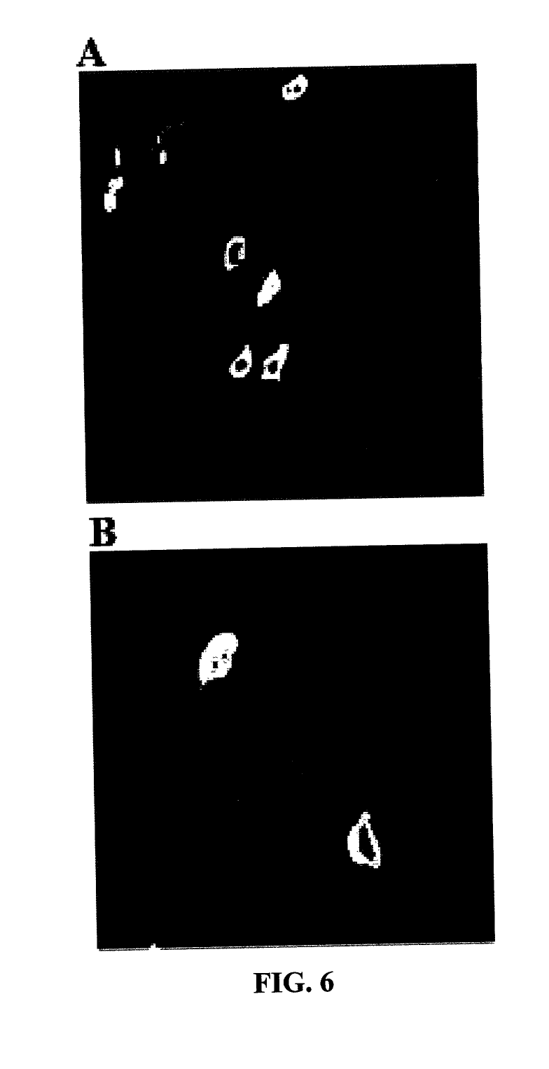

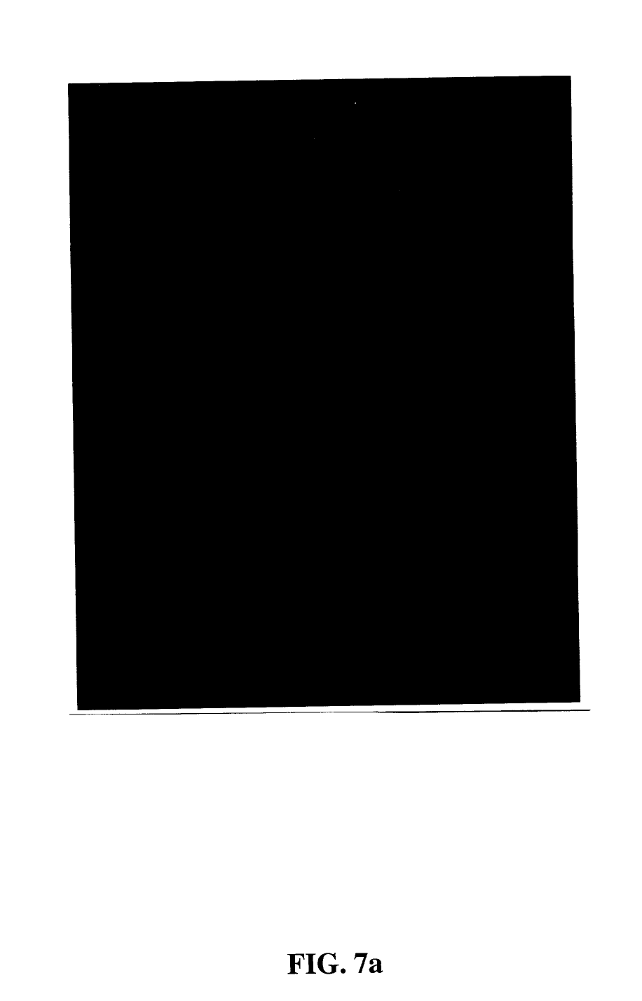

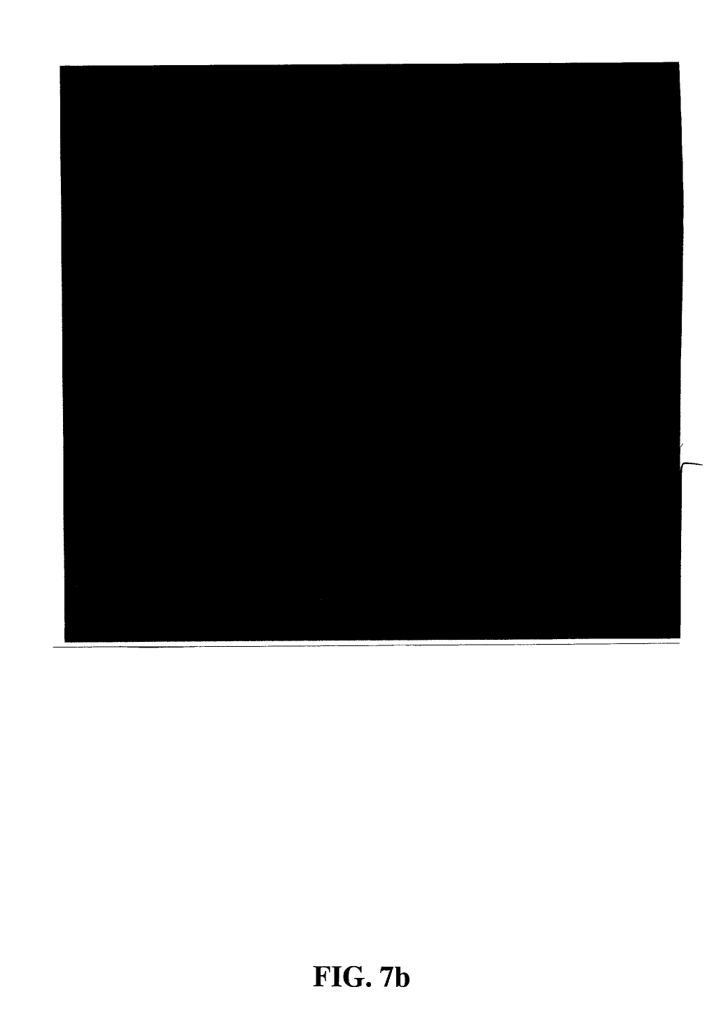

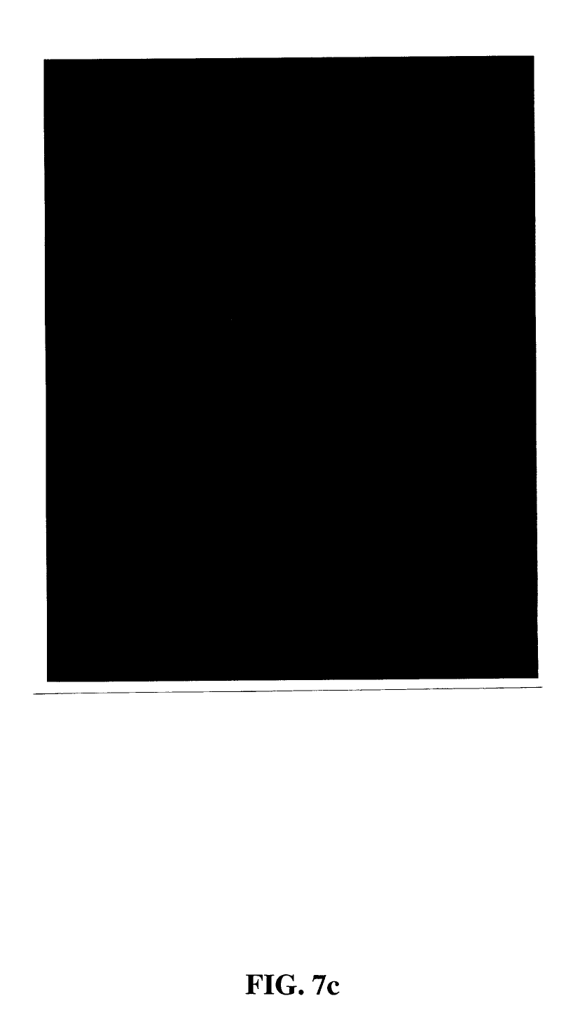

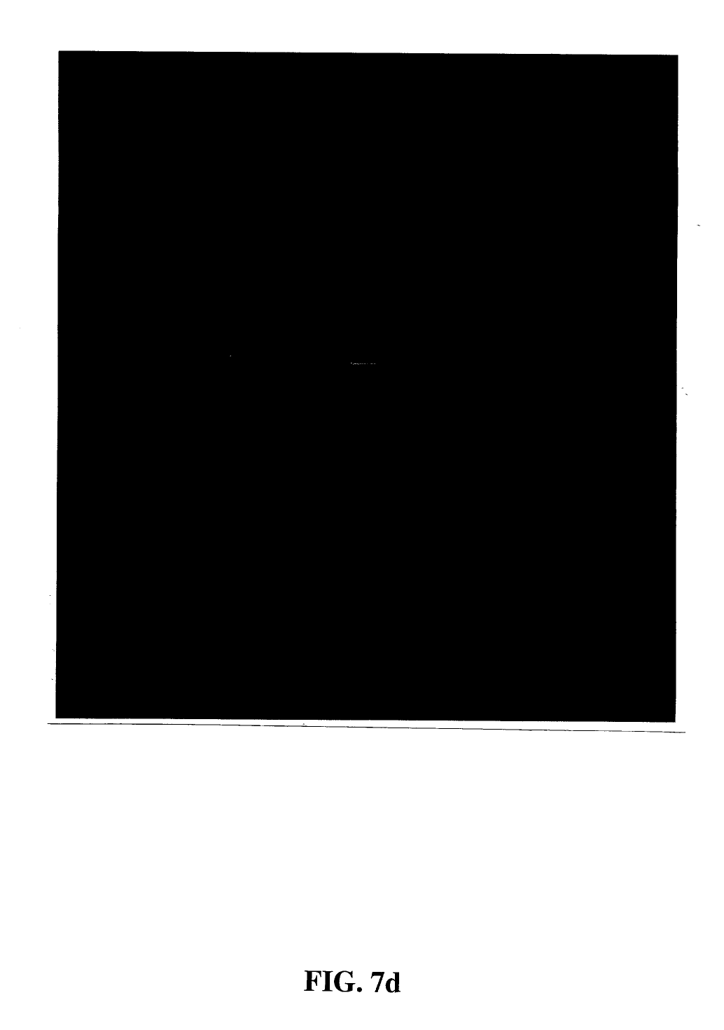

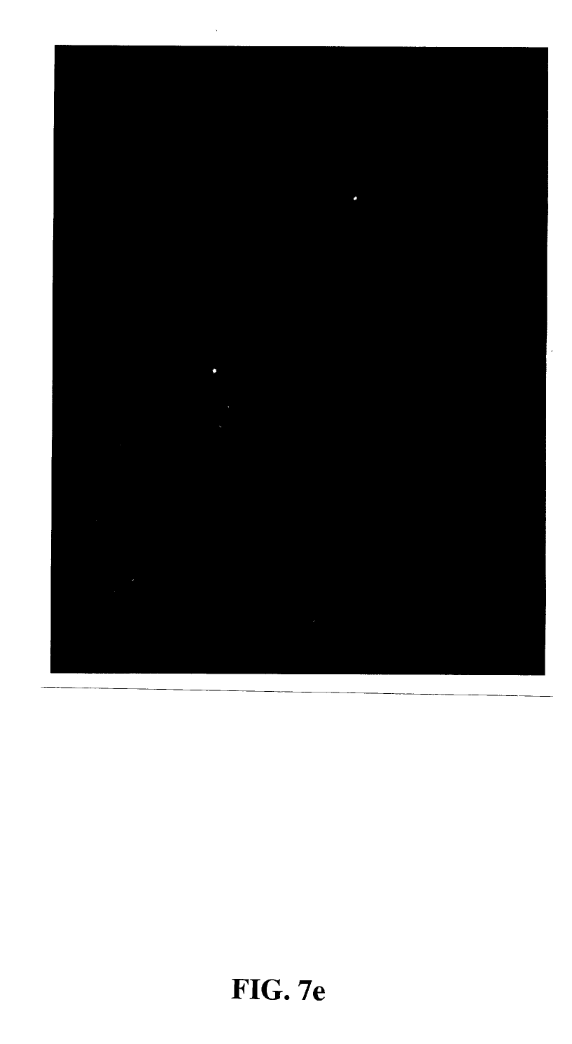

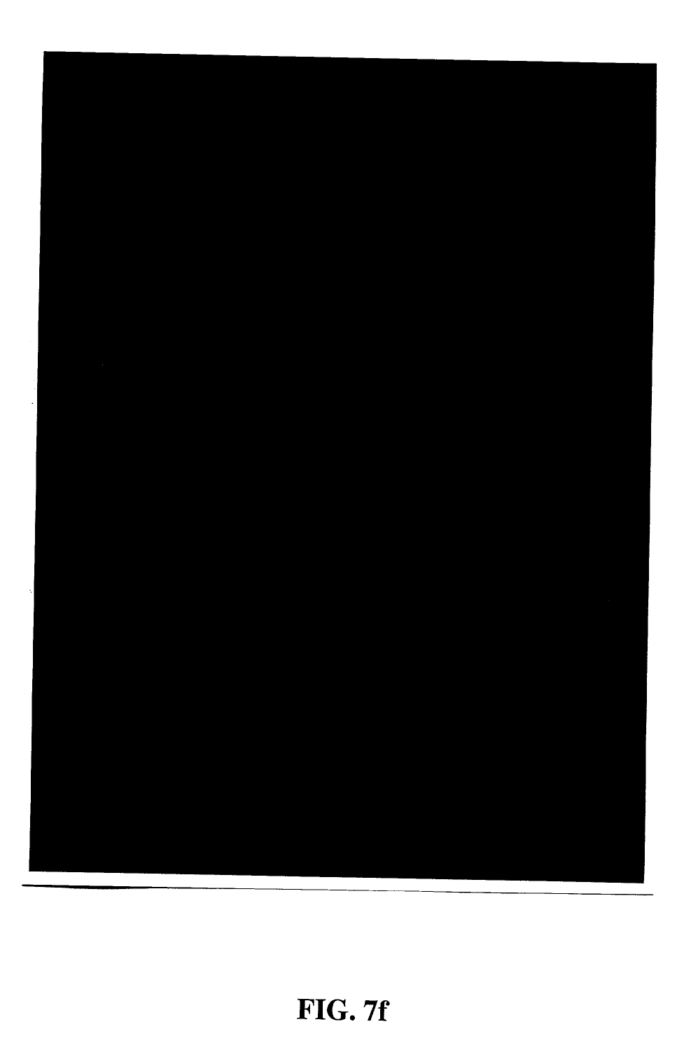

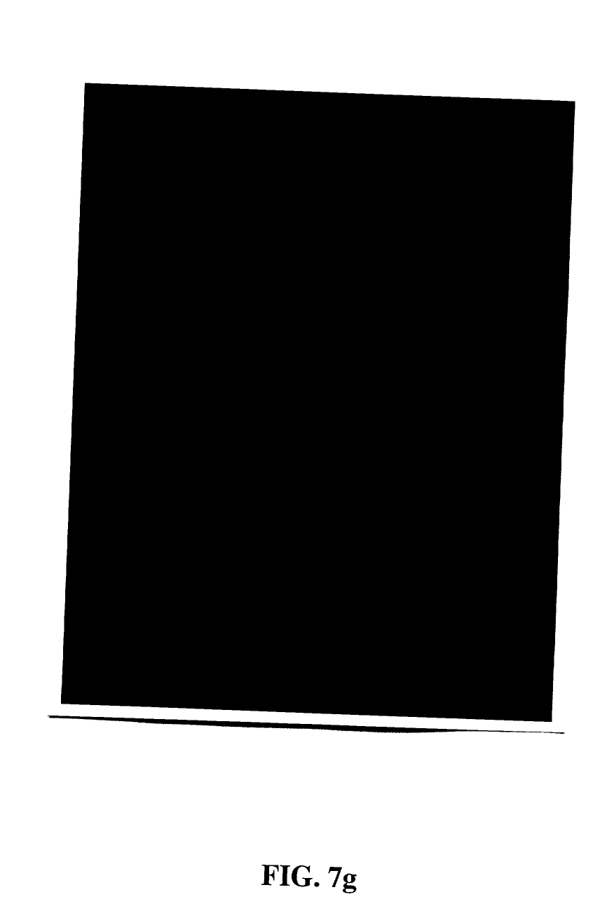

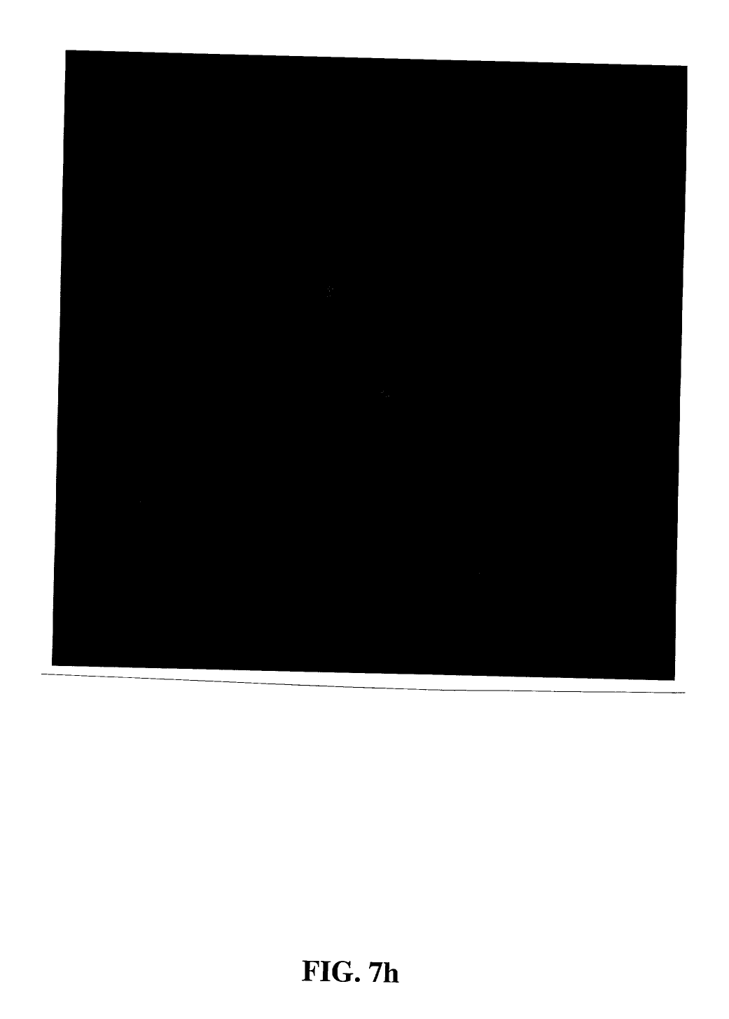

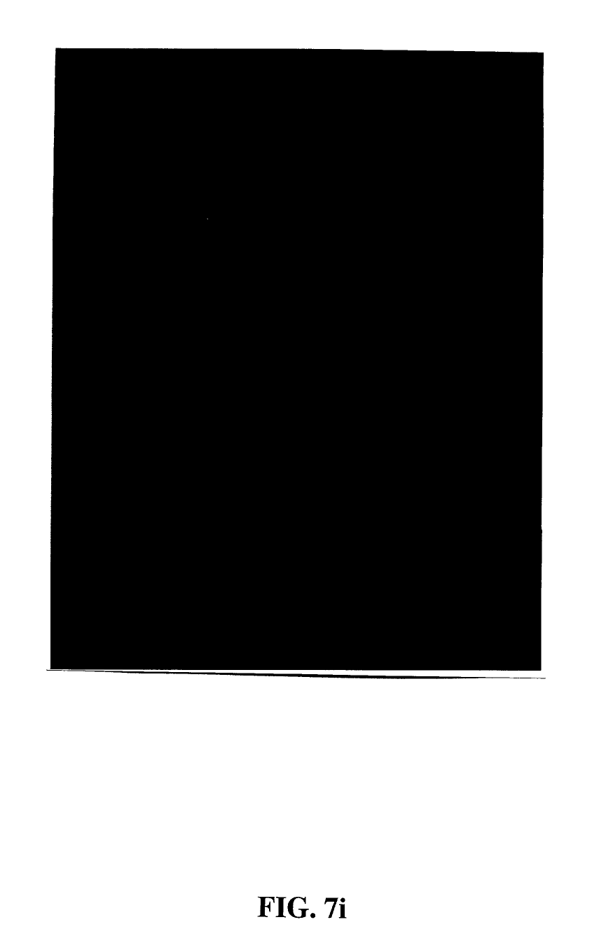

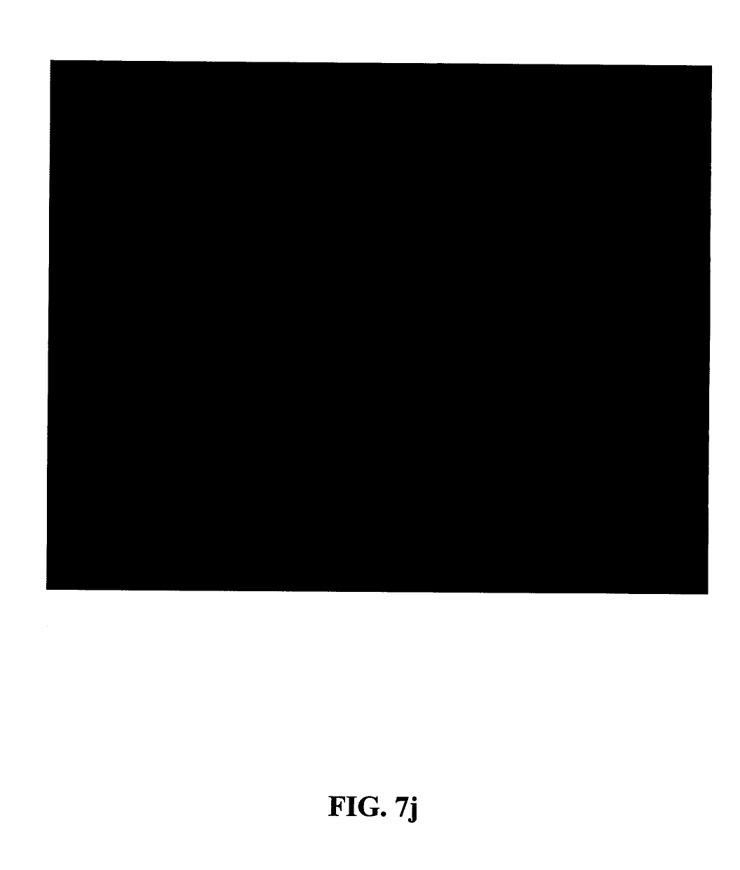

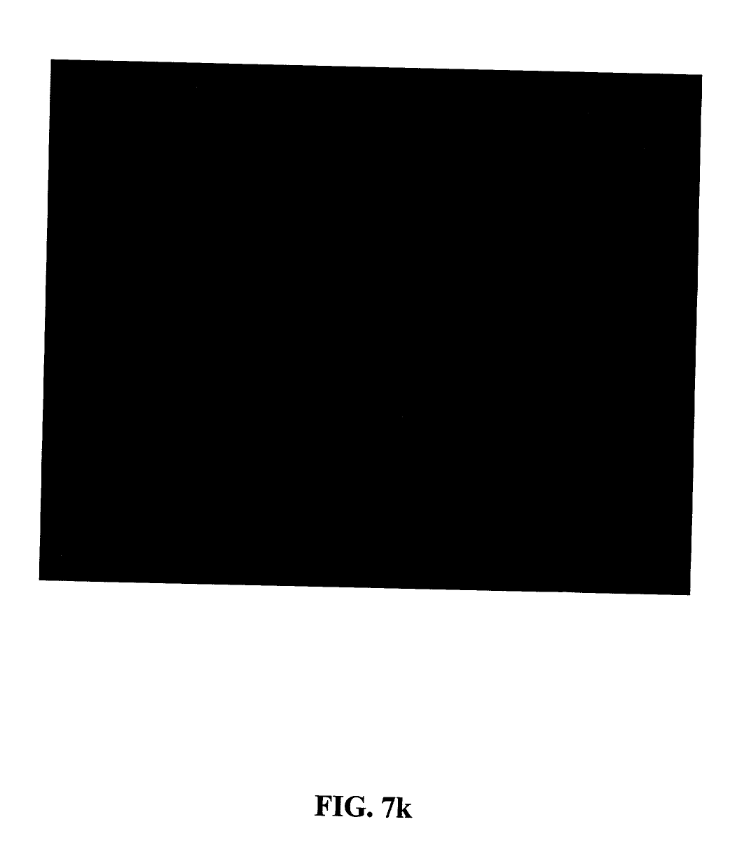

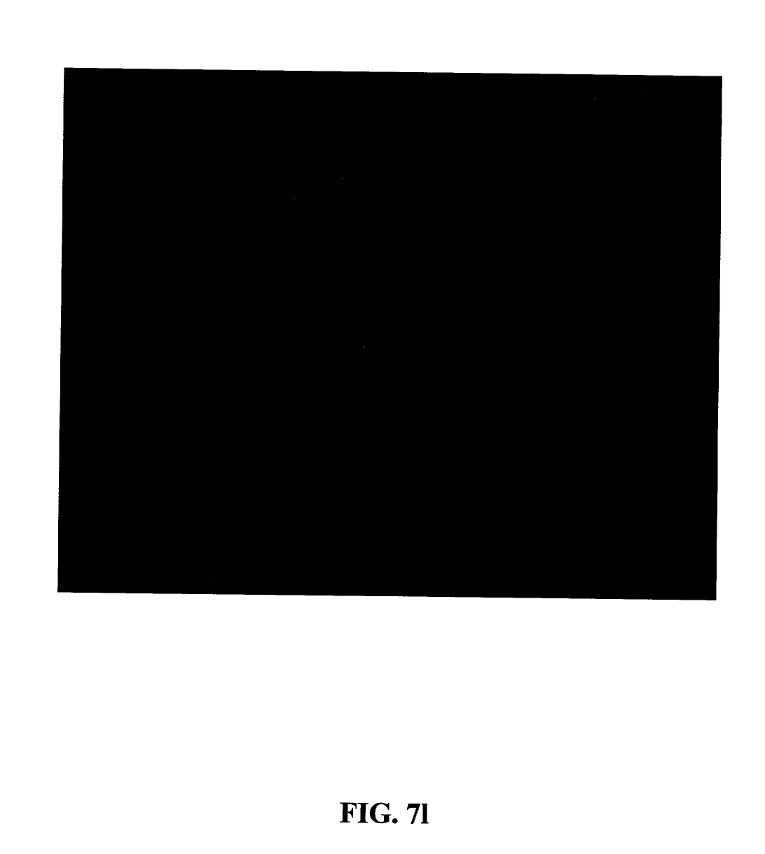

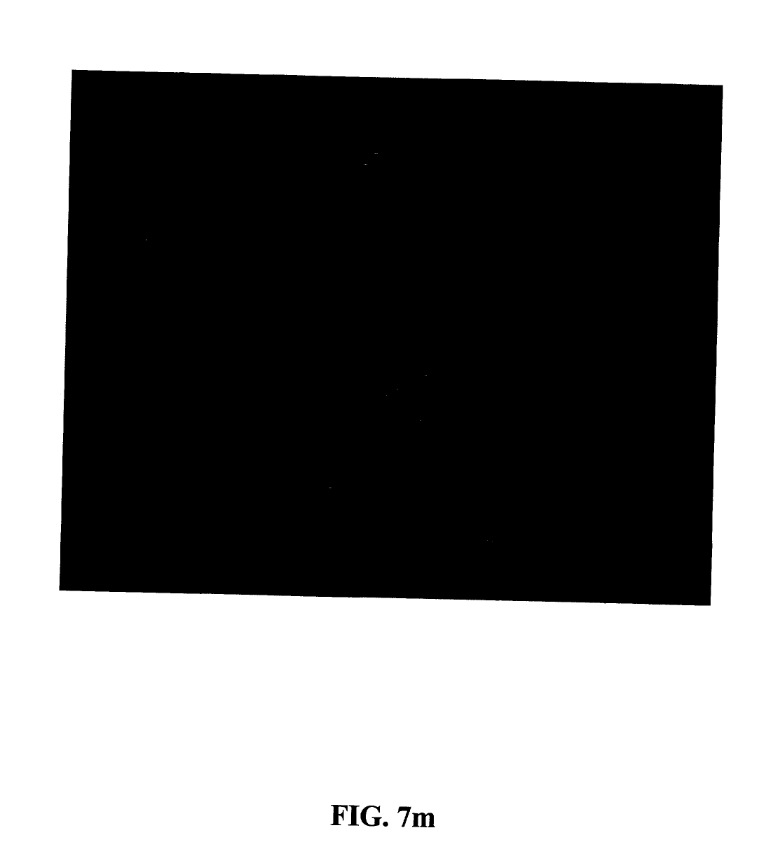

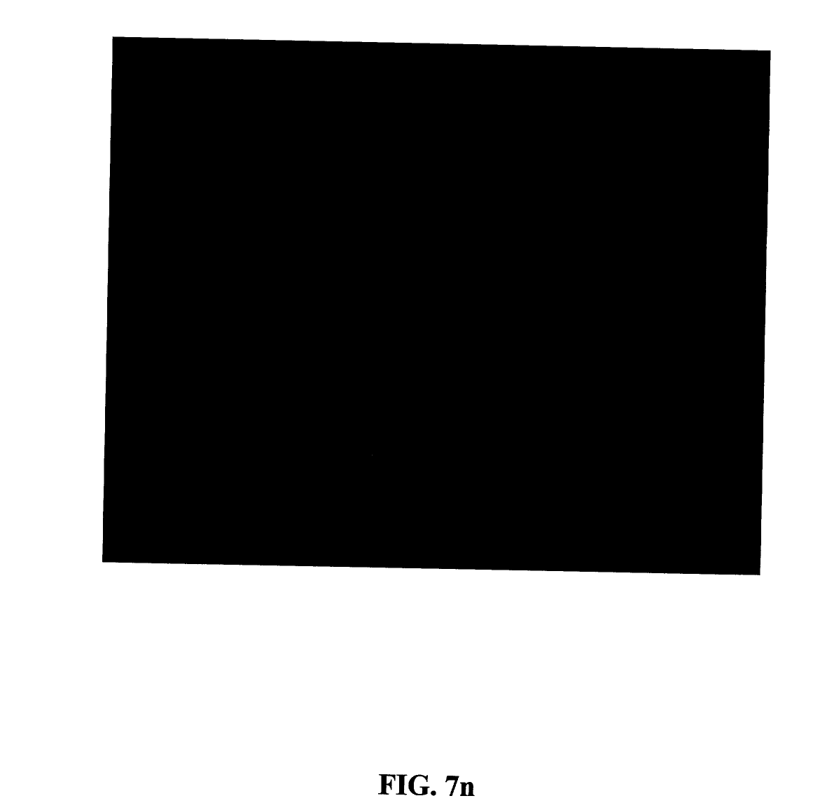

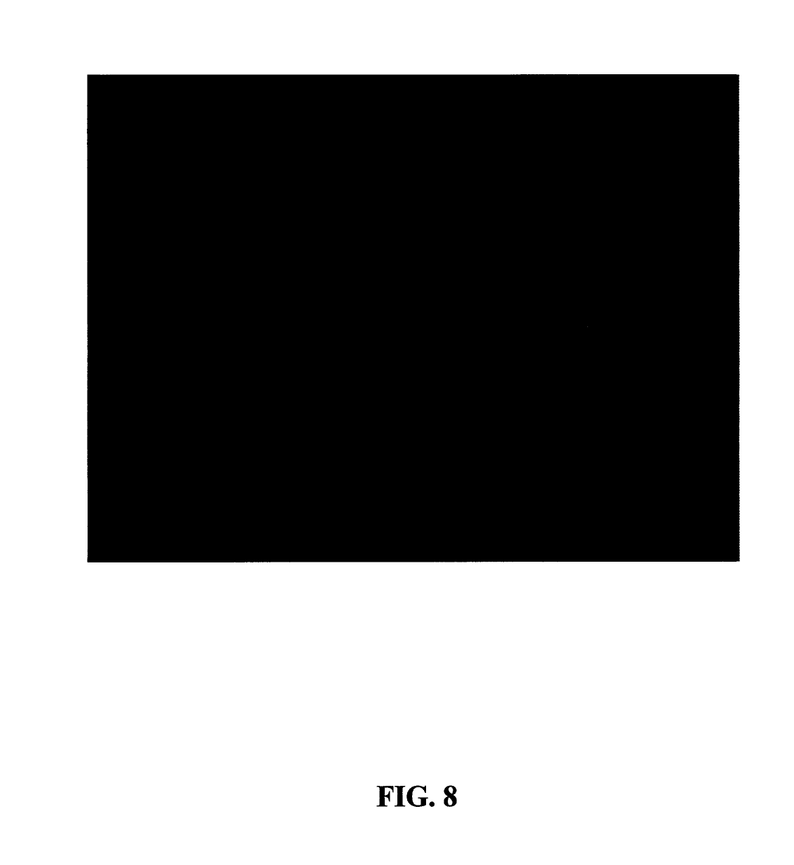

The present invention also provides evidence demonstrating that human stem and progenitor cells, i.e., CD34 cells, extracted from bone marrow, umbilical cord blood, fetal liver tissue, or peripherally mobilized cells that are injected into the CNS of an adult or neonate rat or mouse brain will migrate into multiple target regions, engraft in the CNS, and subsequently differentiate into developmentally and regionally appropriate cells, i.e., neuronal and glial cells. (FIGS. 2-7) Progenitor human cells injected into the brain ventricles or subcortically are widely detected throughout the brain. Most importantly, the cells show a natural ability to migrate away from the injection site, travelling preferentially to lesioned areas in all lesion models (i.e. lysolecithin, NMDA, 6-OHDA). (FIGS. 2-7) The results of the present invention further demonstrate that GFP-transfected stem cells (using AAV-GFP) also migrate and differentiate at a similar rate as non-transfected stem cells. (FIG. 8). Stem and progenitor cells may be driven to migrate to injured areas by factors that are currently still unknown.

The surgical procedure used in the present invention does not cause any systemic or local adverse effect. Blood markers for hematology and chemistry remain within the normal range after surgery. Immunocytochemical analysis using inflammatory and astrocytic markers (i.e. GFAP, CD45, CD3, and CD8) reveals that such markers are absent, thereby evidencing the lack of an inflammatory response. In light of these results, hematopoietic stem and progenitor cell transplantion using autologous or allogeneic cells for brain cell regeneration has enormous therapeutic potential.

We claim:

1. A method of targeted delivery of mammalian stem cells of myeloid origin into a nervous system of a mammal, comprising
(a) administering a therapeutically effective amount of mammalian stem cells of myeloid origin into a nervous system of said mammal;
(b) migrating of said mammalian stem cells of myeloid origin from the injection site to a preferred site in a nervous system of said mammal; and
(c) engrafting of said mammalian stem cells of myeloid origin into said nervous system of said mammal at said preferred site.

2. The method of claim 1, wherein said mammalian stem cells of myeloid origin are derived from at least one of the group of bone marrow, mobilized peripheral blood, umbilical cord blood, or fetal liver tissue from a mammal.

3. The method of claim 1, wherein administration of said therapeutically effective amount of mammalian stem cells is at least one of the group of intrathecal, intraventricular, intracisternal, intraparenchymal into the brain or spinal cord, or systemic.

4. The method of claim 1, wherein administration of said mammalian stem cells of myeloid origin is a combination of at least two of the group of intrathecal, intraventricular, intracisternal, intraparenchymal into the brain or spinal cord, or systemic.

5. The method of claim 1, wherein said mammalian stem cells of myeloid origin maintain the pluripotential capacity to differentiate into neuronal and glial cells.

6. The method of claim 1, wherein said mammalian stem cells are transiently or stably genetically engineered by at least one viral vector or non-viral transfection.

7. The method of claim 1, wherein said mammalian stem cells of myeloid origin deliver viral vectors, other transducing agents, or biological pumps of peptides directly into said nervous system of said mammal.

8. The method of claim 1, wherein delivery of said mammalian stem cells of myeloid origin comprises delivery of cells expressing CD34.

9. The method of claim 1, wherein delivery of said mammalian stem cells of myeloid origin comprises delivery of cells negative for CD34.

10. A method of treating disorders, diseases, or trauma of a nervous system of a mammal, comprising
(a) administering a therapeutically effective amount of mammalian stem cells of myeloid origin into a nervous system of said mammal;
(b) migrating of said mammalian stem cells of myeloid origin from the injection site to a preferred site in a nervous system of said mammal;
(c) engrafting of said mammalian stem cells of myeloid origin into said nervous system of said mammal at said preferred site;
(d) differentiating of said engrafted mammalian stem cells of myeloid origin of step (c) into neuronal and glial cells; and
(e) replacing damaged nervous sytstem tissue of said mammal with said neuronal and glial cells of step (d).

11. The method of claim 10, wherein said mammalian stem cells of myeloid origin are derived from at least one of the group of bone marrow, mobilized peripheral blood, umbilical cord blood, or fetal liver tissue from a mammal.

12. The method of claim 10, wherein administration of said therapeutically effective amount of mammalian stem cells is at least one of the group of intrathecal, intraventricular, intracisternal, intraparenchymal into the brain or spinal cord, or systemic.

13. The method of claim 10, wherein administration of said therapeutically effective amount of mammalian stem cells is a combination of at least two of the group of intrathecal, intraventricular, intracisternal, intraparenchymal into the brain or spinal cord, or systemic.

14. The method of claim 10, wherein said mammalian stem cells are transiently or stably genetically engineered by at least one viral vector or non-viral transfection.

15. The method of claim 10, wherein said mammalian stem cells of myeloid origin deliver viral vectors, other transducing agents, or biological pumps of peptides directly into said nervous system of said mammal.

16. The method of claim 10, wherein administration of said therapeutically effective amount of mammalian stem cells of myeloid origin comprises delivery of cells expressing CD34.

17. The method of claim 10, wherein administration of said therapeutically effective amount of mammalian stem cells of myeloid origin comprises delivery of cells negative for CD34.

18. A method of treating a nervous system disorder, disease, or trauma in a mammal, comprising
(a) administering a therapeutically effective amount of mammalian stem cells of myeloid origin into a nervous system of said mammal, wherein said mammalian stem cells are transiently or stably genetically engineered by at least one viral vector or by non-viral transfection;
(b) migrating said mammalian stem cells of myeloid origin from the injection site to a preferred site in a nervous system of said mammal;
(c) engrafting said mammalian stem cells of myeloid origin into said nervous system of said mammal at said preferred site;
(d) differentiating said engrafted mammalian stem cells of myeloid origin of step (c) into neuronal and glial cells; and
(e) replacing damaged nervous sytstem tissue of said mammal with said neuronal and glial cells of step (d).

19. A method of treating a nervous system disorder, disease, or trauma in a mammal, comprising
(a) administering a therapeutically effective amount of mammalian stem cells of myeloid origin into a nervous system of said mammal, wherein said stem cells of myeloid origin deliver viral vectors, other transducing agents, or biological pumps of peptides directly into said nervous system of said mammal;
(b) migrating said mammalian stem cells of myeloid origin from the injection site to a preferred site in a nervous system of said mammal;
(c) engrafting said mammalian stem cells of myeloid origin into said nervous system of said mammal at said preferred site;
(d) differentiating said engrafted mammalian stem cells of myeloid origin of step (c) into neuronal and glial cells; and
(e) replacing damaged nervous sytstem tissue of said mammal with said neuronal and glial cells of step (d).
